(19) United States
(12) Patent Application Publication
(10) Pub. No.: US 2007/0149395 A1
(43) Pub. Date: Jun. 28, 2007

(54) ZINC OXIDE-CERIUM OXIDE COMPOSITE PARTICLES

(75) Inventors: Michael Kroell, Gelnhausen (DE); Stipan Katusic, Kelkheim (DE); Guido Zimmermann, Rodenbach (DE); Stefan Heberer, Gelnhausen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 11/610,031

(22) Filed: Dec. 13, 2006

(30) Foreign Application Priority Data
Dec. 13, 2005 (DE) 10 2005 059 405.0

(51) Int. Cl. B01J 23/10; A61K 8/00; C04B 14/00

(52) U.S. Cl. 502/304; 424/59; 106/425; 252/588

(57) ABSTRACT

Composite particles useful for absorbing in the UV-A and UV-B regions contain a zinc oxide matrix and cerium oxide domains, wherein the domains are located in and on the matrix, wherein a fraction of zinc oxide is 80 to 98% by weight and a fraction of cerium oxide is 2 to 20% by weight, in each case based on the composite particles, and wherein a BET surface area of said composite particles is from 5 to 100 m²/g.

### BACKGROUND OF THE INVENTION

1. Field of the Invention

The present invention relates to zinc oxide-cerium oxide composite particles, their preparation and use.

2. Description of the Related Art

It is known to reduce the photocatalytic activity of inorganic UV filters, such as titanium dioxide (UV-B) and zinc oxide (UV-A), by coating with a photocatalytically inactive component.

U.S. Pat. No. 6,132,743 discloses zinc oxide particles whose photocatalytic activity is reduced by coating with a silicone compound.

U.S. Pat. No. 6,500,415 discloses titanium dioxide or zinc oxide particles which are coated with silicon dioxide or aluminium oxide in order to minimize their photocatalytic activity.

WO 03/037994 discloses, for example, titanium dioxide particles whose photocatalytic activity can be reduced by coating with an oxide, hydroxide or an oxide hydroxide of aluminium, cerium, zirconium and/or silicon. In the process disclosed in WO 03/037994, a precursor of the coating material is applied to titanium dioxide particles by means of an enzymatic precipitant system. The coated particles are not aggregated and have an average particle size of less than 50 nm. Complete coating is required as an essential feature for a noticeable reduction in the photocatalytic activity.

In summary, the prior art discloses UV-B filters whose photocatalytic activity can be reduced by coating with a photocatalytically inactive component. As a rule, the coating reduces the UV absorption of the coated substance, at best it remains the same. The wavelength of the UV absorption is unchanged or changed only slightly by the coating substances specified in the prior art. The coating thus merely has the task of minimizing the photocatalytic activity without adversely affecting the absorption of the UV filter in the UV-B region too much.

### SUMMARY OF THE INVENTION

It is an object of the present invention to provide a UV filter with only low or no photocatalytic activity which absorbs in the UV-B and U-A regions.

This and other objects have been achieved by the present invention the first embodiment of which includes composite particles, comprising:

a zinc oxide matrix and cerium oxide domains,

wherein the domains are located in and on the matrix,

wherein a fraction of zinc oxide is 80 to 98% by weight and a fraction of cerium oxide is 2 to 20% by weight, in each case based on the composite particles, and

wherein a BET surface area of said composite particles is from 5 to 100 m²/g.

In another embodiment, the present invention provides a process for the preparation of the above composite particles, comprising:

atomizing a solution 1 and a solution 2 with an atomizing gas using a nozzle into a reaction space resulting in the formation of drops having an average diameter of less than 100 μm,

introducing the atomized solutions into a high-temperature zone of a reactor, and reacting said atomized solutions in said high-temperature zone at temperatures of from 800 to 1200° C. with oxygen or an oxygen-containing gas, thereby obtaining hot gases and a solid product,

cooling the hot gases and the solid product, and

separating off the solid product from the gases,

wherein said solution 1 comprises an oxidizable zinc compound, and said solution 2 comprises an oxidizable cerium compound,

wherein a fraction of solution 1 is 80 to 98% by weight, calculated as ZnO, and a fraction of solution 2 is 2 to 20% by weight, calculated as CeO₂,

wherein solution 1 has a viscosity of from 200 to 5000 mPas and solution 2 has a viscosity of from 5 to 150 mPas, and optionally, the viscosity is regulated by heating to temperatures in each case within the decomposition temperature of the zinc compound and of the cerium compound.

### BRIEF DESCRIPTION OF DRAWINGS

FIG. 1 shows a transmission electron micrograph of the composite particles of the present invention.

FIG. 2 shows the absorbance of ZnO (......), CeO₂ (----) and the composite particles according to the present invention (------) (solid line) as a function of the wavelength in nm.

### DETAILED DESCRIPTION OF THE INVENTION

The present invention provides composite particles with a BET surface area of from 5 to 100 m²/g comprising a zinc oxide matrix and cerium oxide domains, where the domains are located in and on the matrix and the fraction of zinc oxide is 80 to 98% by weight and the fraction of cerium oxide is 2 to 20% by weight, in each case based on the composite particles.

The BET surface area includes all values and subvalues therebetween, especially including 10, 15, 20, 25, 30, 35, 40, 45, 50, 55, 60, 65, 70, 75, 80, 85, 90 and 95 m²/g. The fraction of zinc oxide includes all values and subvalues therebetween, especially including 82, 84, 86, 88, 90, 92, 94 and 96% by weight. The fraction of cerium oxide includes all values and subvalues therebetween, especially including 4, 6, 8, 10, 12, 14, 16 and 18% by weight.

The composite particles according to the present invention can be in aggregated form or as isolated individual particles. Aggregated composite particles have a high absorbance in the UV-A and UV-B regions and exhibit low photocatalytic activity. The composite particles according to the present invention are therefore preferably in aggregated form.

The particles according to the present invention have a zinc oxide fraction of from 80 to 98% by weight and a cerium oxide faction of from 2 to 20% by weight. With a fraction of less than 2% by weight, a noteworthy reduction in the photocatalytic activity compared to pure zinc oxide particles can still not be established. With cerium oxide fractions of more than 20% by weight, further reduction in the photocatalytic activity cannot be established.

Preferably, the zinc oxide fraction is 85 to 95% by weight and the cerium oxide fraction is 5 to 15% by weight.

In the composite particles according to the present invention, the sum of the fractions of zinc oxide and cerium oxide is preferably at least 99.9% by weight, based on the composite particles. In particular, it may be advantageous to reduce the fraction of metallic impurities. Thus, composite particles may be particularly advantageous whose fractions of lead are less than 20 ppm, of arsenic less than 3 ppm, of cadmium less than 15 ppm, of mercury less than 1 ppm, of iron less than 200 ppm and of antimony less than 1 ppm, in each case based on the composite particles.

The BET surface area of the composite particles is preferably 20 to 50 m²/g. Within this range, the composite particles have high UV-A and UV-B absorption coupled with low photoactivity.

The matrix of the composite particles preferably has an average diameter of from 20 to 100 nm. Within this range, the composite particles have high UV-A and UV-B absorption coupled with low photoactivity. The average diameter of the matrix of the composite particles includes all values and subvalues therebetween, especially including 25, 30, 35,40, 45, 50, 55, 60, 65, 70 5, 80, 85, 90 and 95 nm.

The domains of the composite particles preferably have an average diameter of from 2 to 10 nm. Within this range, the composite particles have high UV-A and UV-B absorption coupled with low photoactivity. The average diameter of the domains includes all values and subvalues therebetween, especially including 4, 6 and 8 nm.

The present invention further provides a process in which
a solution 1, which comprises an oxidizable zinc compound, and a solution 2, which comprises an oxidizable cerium compound, are atomized with an atomizing gas by means of a nozzle into a reaction space with the formation of drops having an average diameter of less than 100 μm, where
the solvents are preferably of an organic nature,
the fraction of solution 1 is 80 to 98% by weight, calculated as ZnO, and the fraction of solution 2 is 2 to 20% by weight, calculated as CeO₂,
solution 1 has a viscosity of from 200 to 5000 mPas and solution 2 has a viscosity of from 5 to 150 mPas, and the viscosity is regulated, if appropriate, by heating to temperatures in each case within the decomposition temperature of the zinc compound and of the cerium compound,
the atomized solutions are introduced into a high-temperature zone of a reactor, and are reacted there at temperatures of from 800 to 1200° C. with oxygen or an oxygen-containing gas,
the hot gases and the solid product are cooled and then the solid product is separated off from the gases.

The average diameter of the drops includes all values and subvalues therebetween, especially including 5, 10, 15, 20, 25, 30, 35, 40, 45, 50, 55, 60, 65, 70, 75, 80, 85, 90 and 95 μm. The fraction of solution 1 includes all values and subvalues therebetween, especially including 82, 84, 86, 88, 90, 92, 94 and 96% by weight. The fraction of solution 2 includes all values and subvalues therebetween, especially including 4, 6, 8, 10, 12, 14, 16 and 18% by weight. The viscosity of solution 1 includes all values and subvalues therebetween, especially including 300, 400, 500, 600, 700, 800, 900, 1000, 1500, 2000, 2500, 3000, 3500, 4000 and 4500 mPas. The viscosity of solution 2 includes all values and subvalues therebetween, especially including 10, 15, 20, 25, 30, 35, 40, 45, 50, 55, 60, 65, 70, 75, 80, 85, 90, 95, 100, 105, 110, 115, 120, 125, 130, 135, 140 and 145 mPas. The reaction temperature includes all values and subvalues therebetween, especially including 850, 900, 950, 1000, 1050, 1100 and 1150° C.

Preferably, the temperature required in the high-temperature zone is produced with a flame which is obtained from the reaction of a hydrogen-containing combustion gas with oxygen and or air. The temperature can be adjusted through the ratio of hydrogen-containing combustion gas and oxygen. Suitable hydrogen-containing combustion gases may be: hydrogen, methane, ethane, propane, butane and or natural gas. Preference is given to using hydrogen.

The present invention further provides a dispersion which comprises the composite particles according to the present invention. The content of composite particles may be 0.1 to 60% by weight, preferably from 10 to 40% by weight, based on the total amount of the dispersion. The content of composite particles includes all values and subvalues therebetween, especially including 0.5, 1, 5, 10, 15, 20, 25, 30, 35, 40, 45, 50 and 55% by weight. It can either be aqueous or organic, or consist of a mixture which has water and organic solvents as liquid phase, where, in all cases, only a single liquid phase is present. Aqueous is understood as meaning that the majority of the liquid phase consists of water. Organic is to be understood as meaning that the liquid phase consists predominantly or exclusively of at least one organic solvent. Suitable organic solvents may be mono, di-, tri, polyalcohols, ethers, esters, aromatics, alkanes and alkenes. In particular, ethanol, methanol, propanol, butanol, acetone, ethyl acetate, and butyl acetate can be used. The organic solvent may also be a reactive thinner, such as, for example, hexanediol diacrylate or tripropylene glycol diacrylate.

The dispersion according to the present invention can also comprise additives. These may be a dispersion auxiliary, an emulsifier, a pH regulating substance and/or a stabilizer. Preferably, this may be Na polyphosphate, ascorbic acid, citric acid, 6 aminohexanoic acid, stearic acid and/or salts of polyacrylic acid, in particular the sodium salt. In addition, Disperbyk 163, Disperbyk 180, Disperbyk 190 and/or Byk 9077 can be used. The additive is preferably present in an amount of from 0.1 to 5% by weight, particularly preferably from 0.5 to 1.5% by weight, based on the liquid phase of the dispersion. The amount of additive includes all values and subvalues therebetween, especially including 0.5, 0.6, 0.7, 0.8, 0.9, 1, 1.1, 1.2, 1.3, 1.4, 1.5, 2, 2.5, 3, 3.5, 4 and 4.5% by weight.

The present invention further provides a coating preparation which comprises the composite particles according to the present invention or the dispersion according to the present invention and at least one binder.

Suitable binders may be polyacrylates, polyurethanes, polyalkyds, polyepoxides, polysiloxanes, polyacrylonitriles and/or polyesters. In the case of dispersions which have one or more reactive thinners as liquid phase, an aliphatic urethane acrylate, for example Laromer® LR8987, BASF, may be particularly suitable. The coating preparation according to the present invention can preferably comprise polyacrylates and or polyurethanes.

The fraction of binder in the coating preparation is preferably between 0.1 and 50% by weight. Particular preference is given to a range between 1 and 10% by weight. The fraction of binder includes all values and subvalues therebetween, especially including 0.5, 1, 5, 10, 15, 20, 25, 30, 35, 40 and 45% by weight.

The fraction of composite particles in the coating preparation is preferably between 0.1 and 60% by weight. Particular preference is given to a range between 1 and 10% by weight. The fraction of composite particles includes all values and subvalues therebetween, especially including 0.5, 1, 5, 10, 15, 20, 25, 30, 35, 40, 45, 50 and 55% by weight.

Furthermore, the coating preparation can, during applications, comprise compounds for changing the rheology of the coating preparation. Fillers containing silicon dioxide are particularly advantageous, with pyrogenic silicon dioxide being particularly preferred. The amount may preferably be between 0.1 and 20% by weight, based on the total coating preparation. The amount of fillers includes all values and subvalues therebetween, especially including 0.5, 1, 5, 10 and 15% by weight.

In addition, the coating preparation can comprise organic solvents, such as ethanol, butyl acetate, ethyl acetate, acetone, butanol, THF, alkanes or mixtures of two or more of these specified substances in amounts of from 1% by weight to 98% by weight, based on the total coating preparation. The amount of solvent includes all values and subvalues therebetween, especially including 5, 10, 15, 20, 25, 30, 35, 40,45, 50, 55, 60 65, 70, 75, 80, 85, 90 and 95% by weight.

The coating preparation according to the present invention can be used for the coating of substrates made of wood, PVC, plastic, steel, aluminium, zinc, copper, MDF, glass and concrete.

The present invention further provides a sunscreen formulation which comprises the composite particles according to the present invention.

The fraction of composite particles can preferably be 0.01 to 25% by weight, based on the sunscreen formulation. The fraction of composite particles includes all values and subvalues therebetween, especially including 0.05, 0.1, 0.5, 1, 5, 10, 15 and 20% by weight.

In addition, the sunscreen composition according to the present invention can be used in mixtures with known inorganic UV-absorbing pigments and/or chemical UV filters. Suitable known UV-absorbing pigments are titanium dioxides, zinc oxides, aluminium oxides, iron oxides, silicon dioxide, silicates, cerium oxides, zirconium oxides, barium sulphate or mixtures thereof Suitable chemical UV filters are all water-soluble or oil-soluble UVA and UV-B filters known to the person skilled in the art, of which mention may be made, in an exemplary but nonlimiting way, of sulphonic acid derivatives of benzophenones and benzimidazoles, derivatives of dibenzoylmethane, benzylidenecamphor and derivatives thereof, derivatives of cinnamic acid and esters thereof, or esters of salicylic acid. The sunscreen compositions according to the present invention may also comprise the solvents known to the person skilled in the art, such as water, mono- or polyhydric alcohols, cosmetic oils, emulsifiers, stabilizers, consistency regulators, such as carbomers, cellulose derivatives xanthan gum, waxes, bentones, pyrogenic silicas and further substances customary in cosmetics, such as vitamins, antioxidants, preservatives, dyes and perfumes.

Typically, the sunscreen composition according to the present invention can be present as an emulsion (O/W, W/O or multiple), aqueous or aqueous-alcoholic gel or oil gel, and be supplied in the form of lotions, creams, milk sprays, mousse, stick or in other customary forms.

The present invention further provides the use of the composite particles according to the present invention, of the dispersion according to the present invention, of the coating composition according to the present invention or of the sunscreen formulation according to the present invention as UV filter.

Having generally described this invention, a further understanding can be obtained by reference to certain specific examples which are provided herein for purposes of illustration only, and are not intended to be limiting unless otherwise specified.

### EXAMPLES

### Materials Used

Solution A: Octa-Soligen Zinc 10 (Borchers):
Viscosity (20° C.): 325 mPas
Zinc octoate: 10% by weight, calculated as zinc
Test benzene: 42% by weight
C₆-C₁₉ fatty acids: 46% by weight
2-(2-Butoxyethoxyethanol): 2% by weight
Solution B: Borchi Kat 22, Borchers,
Viscosity (20° C.): 5017 mPas
Zinc octoate: 22% by weight, calculated as zinc
C₆-C₁₉ fatty acids: 78% by weight
Solution C: Octa-Soligen Cerium 12, Borchers,
Viscosity (30° C.): 61 mPas
Cerium octoate 50% by weight
2-Ethylhexanoic acid 50% by weight.
Solution D: Cerium(III) nitrate hexahydrate dissolved in acetone, content of cerium 8% by weight, viscosity (20° C.): 52 mPas

### Example 1

1463 g/h of solution A with a temperature of 20° C., which, at this temperature, had a viscosity of 325 mPas, and 136 g/h of solution C with a temperature of 80° C., which, at this temperature, had a viscosity of 9.2 mPas, were atomized by means of a triple-material nozzle into a reaction space with formation of an aerosol. The average drop diameter D₃₀ was less than 100 μm. Here, an oxyhydrogen gas flame comprising hydrogen (3 Nm³/h) and primary air (5 Nm³/h) burns, in which the aerosol was reacted. Additionally secondary air (25 Nm³) was introduced into the reaction space. The temperature 0.5 m below the flame was 925° C. The reaction mixture was then cooled and the solid product was separated off from gaseous substances over a filter.

Examples 2 to 6 were carried out analogously. Feed materials and amounts used are shown in Table 1. Composition and BET surface area of the composite particles obtained are likewise shown in Table 1.

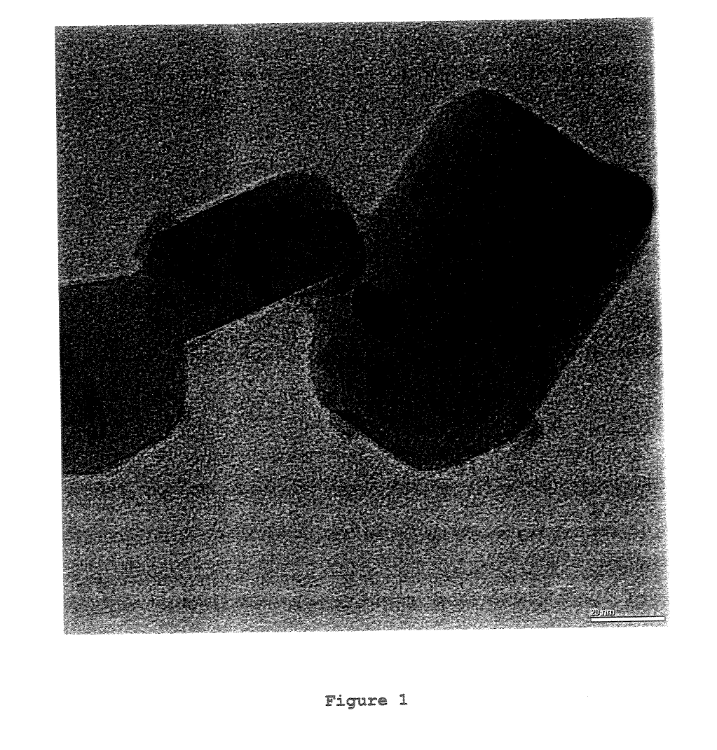

FIG. 1 shows a transmission electron micrograph of the composite particles from Example 2. The darker cerium oxide domains in a zinc oxide matrix can clearly be seen. The diameter of the zinc oxide matrix was generally 20 to 100 nm and that of the cerium oxide domains was generally 5 to 10 nm. The zinc oxide faction was in hexagonal form, and the cerium oxide faction was in cubic form.

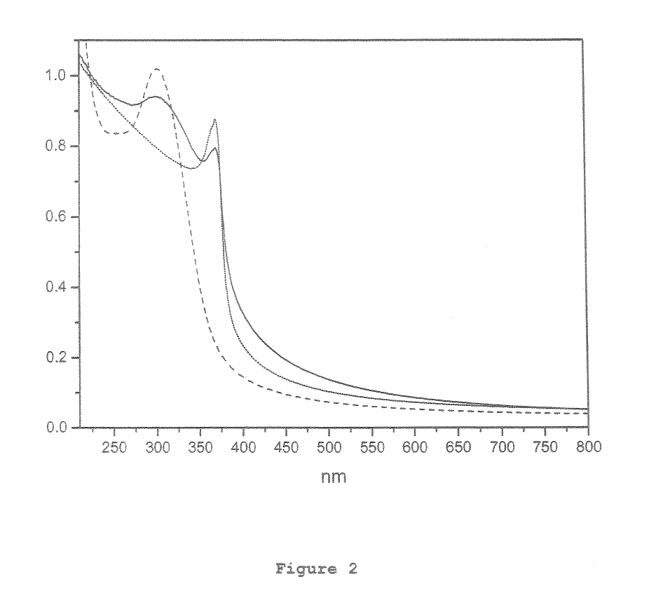

FIG. 2 shows the absorbance of ZnO (......) CeO₂ (----) and the composite particles according to the present invention from Example 2 (------) (solid line) as a function of the wavelength in nm. In the case of the composite particles according to the present invention, which comprise about 90% by weight of zinc oxide and about 10% by weight of cerium oxide, it can clearly be seen that the absorbance was only slightly less despite the low faction of cerium oxide than in the case of pure cerium oxide.

TABLE 1
Feed materials and amounts used; analytical
values of the resulting composite particles

| | | Example | | | | | |
|---|---|---|---|---|---|---|---|
| | | 1 | 2 | 3 | 4 | 5 | 6 |
| Solution A | | | | | | | |
| Concentration | g/h | 1385 | 1463 | — | — | 1461 | 1853 |
| Temperature | ° C. | 23 | 23 | — | — | 23 | 23 |
| Viscosity | mPas | 325 | 325 | — | — | 325 | 325 |
| Solution B | | | | | | | |
| Concentration | g/h | — | — | 1034 | 1057 | — | — |
| Temperature | ° C. | — | — | 23 | 23 | — | — |
| Viscosity | mPas | — | — | 5000 | 5000 | — | — |
| Solution C | | | | | | | |
| Concentration | g/h | 115 | 136 | 166 | 343 | — | — |
| Temperature | ° C. | 80 | 80 | 80 | 80 | — | — |
| Viscosity | mPas | 9.2 | 9.2 | 9.2 | 9.2 | — | — |
| Solution D | | | | | | | |
| Concentration | g/h | — | — | — | — | 139 | 47 |
| Temperature | ° C. | — | — | — | — | 40 | 40 |
| Viscosity | mPas | — | — | — | — | 32 | 32 |
| Hydrogen | Nm³/h | 3 | 3 | 2 | 2 | 3 | 5 |
| Atomizatinon air | Nm³/h | 5 | 5 | 4 | 5 | 5 | 6 |
| Primary air | Nm³/h | 25 | 25 | 20 | 20 | 25 | 20 |
| Secondary air | Nm³/h | 10 | 10 | 10 | 10 | 15 | 15 |
| Temperature | ° C. | 950 | 925 | 900 | 900 | 975 | 1050 |
| ZnO | wt. % | 91 | 90 | 92 | 85 | 93 | 98 |
| CeO₂ | wt. % | 9 | 10 | 8 | 15 | 7 | 2 |
| BET surface area | m²/g | 28 | 36 | 30 | 45 | 26 | 25 |

On the other hand, the absorbance of zinc oxide fractions in the composite particles virtually reaches the absorbance of pure zinc oxide. There was thus a synergistic effect present in that the integral absorbance of the composite particles was greater than would result arithmetically from the sum of the individual components (10% cerium oxide, 90% zinc oxide). The composite particles according to the present invention were further characterized by the fact that their constituents have high absorption in the UV-A and UV-B regions and can thus be used as broadband filters.

### Example 7

Photocatalytic Activity

The photocatalytic activity of zinc oxide (comparison) and the composite particles from Example 2 was carried out by reference to the degradation of dichloroacetic acid (DCA) using an irradiation reactor, thermostatted at 20° C., having a quartz glass window with an area of 4.9 cm² and a volume of 250 ml. Irradiation was with a 450 W xenon lamp (Osram XBO) and an irradiation intensity of 65 mW/cm². The irradiation time was at least 4 hours. The material to be tested was in the form of a 0.1% strength dispersion in water. In order to keep the ionic strength constant throughout the experiment, 10 mM KNO3 were additionally added to the dispersion. The starting concentration of the dichloroacetic acid (DCA) was 1 mM. The pH of the continuously stirred dispersion was kept constant by adding 0.1M NaOH, while 0.1M NaOH was added by titration.

The degradation of the DCA can be monitored directly by reference to the consumption of sodium hydroxide solution for keeping the ph constant. The following stoichiometry applies:
CHCl₂COO⁻+O₂−>H⁺+2 Cl⁻2 CO₂

From the initial increases of the proton formation curves obtained, it was possible to determine the degradation rate (nM/s) and, from this, the photon efficiency (%) based on the irradiated light intensity.

Pure zinc oxide thus gives a DCA degradation rate of 21.5 nM/s and a photon efficiency of 0.58%. The composite particles according to the present invention from Example 2, on the other hand, have a DCA degradation rate of merely 4.23 nM/s and a photon efficiency of 0.11%.

Even with a cerium oxide fraction of only 10% by weight and the fact that the majority of the surface of the composite particles used was zinc oxide, the photocatalytic activity can be reduced significantly. It was also found that the photocatalytic activity can no longer be reduced to a noteworthy extent by fractions of cerium oxide of more than 20% by weight.

### Example 8

Preparation of a Dispersion According to the Present Invention

The composite particles from Example 2 were added in portions, with stirring, to 50 g of water to which 0.1% by weight of polyacrylic acid in the form of the sodium salt had been added, until a solids content of 10% by weight results. The mixture was then dispersed in each case for one minute with an ultrasound finger (diameter: 7 mm, instrument: ultrasound processor UP 400s, power: 400 W, Dr Hielscher).

### Example 9

Preparation of an Acrylic/Polyurethane-Based Coating Preparation According to the Present Invention

The dispersion from Example 8 was added, under dispersing conditions, to a standard commercial acrylic/polyurethane binder preparation (Relius Aqua Siegel Gloss) so that a coating preparation with a fraction of composite particles of 2% by weight results.

### Example 10

Preparation of an Acrylic-Based Coating Preparation According to the Present Invention

Procedure as in Example 9, but using a standard commercial acrylic binder preparation. (Macrynal SM 510 (Cytec), Desmodur N75 (Bayer).

### Example 11

UV Resistance When Coating Wood

The coating preparations from Example 9 and 10 were each used to coat 3 pinewood samples which have been pretreated with a primer (Relius Aqua Holz Grund) (QUV B 313; DIN EN 927-6, ISO 11507, ASTM D 4857). The comparison used was pinewood samples coated with an acrylic/polyurethane-based coating preparation which was free from composite particles (Relius Aqua Siegel Gloss).

After a test time of 1000 hours, the coatings from Examples 9 and 10 exhibited significantly less yellowing, significantly higher gloss and no brittleness or cracking in the coating compared to the coating without composite particles.

### Example 12

Hardness When Coating Glass

The coating preparations from Examples 9 and 10, and an acrylic/polyurethane-based coating preparation without composite particles (Relius Aqua Siegel Gloss) as comparison, were applied to glass plates in a layer thickness of 150 μm. The hardness was determined after drying times of 1, 6, 13 and 34 days under normal laboratory conditions (20° C., 65% relative humidity) (DIN ISO 1522). The hardness of the layers originating from Examples 9 and 10 was up to 100% greater than that of the comparative example.

### Example 13

Gloss and Degree of Whiteness on Coated Metal Plates

The metal plates were pretreated with a white coating. The coating compositions 9 and 10 and a standard commercial coating composition were then treated with an organic UV filter and irradiated for 55 days in accordance with DIN53231.

After this time, the degree of whiteness according to Berger of the sample with organic UV filter was significantly less than that of the samples from the compositions of Examples 9 and 10.

### Example 14

Preparation of a Sunscreen Formulation According to the Present Invention

The formulation below was used to prepare a sunscreen composition with 4% by weight of the particles according to the present invention as in Example 2.

| Phase | Constituent | Wt. % |
|---|---|---|
| A | Isolan GI 34 | 3.0 |
| | Castor oil | 1.2 |
| | Tegesoft OP | 10.0 |
| | Tegesoft Liquid | 5.0 |
| | Glycerol 86% | 3.0 |
| B | Paracera W80 | 1.8 |
| | Isohexadecane | 5.0 |
| C | Composite particles as in Example 2 | 4.0 |
| D | Magnesium sulphate | 0.5 |
| | Demineralized water | 66.5 |

Phase A was heated to 70° C. in a mixer. After melting on a magnetic heating plate at 80° C., phase B was added to phase A. Phase C was stirred into the oil phase at about 300 rpm and under reduced pressure. Phase D was likewise heated to 70° C. and added to the mixture of A-C under reduced pressure.

German patent application 102005059405.0 filed Dec. 13, 2005, is incorporated herein by reference.

Numerous modifications and variations on the present invention are possible in light of the above teachings. It is therefore to be understood that within the scope of the appended claims, the invention may be practiced otherwise than as specifically described herein.

1. Composite particles, comprising:
a zinc oxide matrix and cerium oxide domains,
wherein the domains are located in and on the matrix,
wherein a fraction of zinc oxide is 80 to 98% by weight and a fraction of cerium oxide is 2 to 20% by weight, in each case based on the composite particles, and
wherein a BET surface area of said composite particles is from 5 to 100 m²/g.

2. The composite particles according to claim 1, which are in aggregated form.

3. The composite particles according to claims 1, wherein the zinc oxide fraction is 85 to 95% by weight and the cerium oxide fraction is 5 to 15% by weight, in each case based on the composite particles.

4. The composite particles according to claim 1, wherein the fraction of zinc oxide and cerium oxide is at least 99.9% by weight, based on the composite particles.

5. The composite particles according to claim 1, wherein the BET surface area is 20 to 50 m²/g.

6. The composite particles according to claim 1, wherein the matrix has an average diameter of from 20 to 100 nm.

7. The composite particles according to claim 1, wherein the domains have an average diameter of from 2 to 10 nm.

8. A process for the preparation of the composite particles according to claim 1, comprising:
atomizing a solution 1 and a solution 2 with a atomizing gas using a nozzle into a reaction space resulting in the formation of drops having an average diameter of less than 100 μm,
introducing the atomized solutions into a high-temperature zone of a reactor, and reacting said atomized solutions in said high-temperature zone at temperatures of from 800 to 1200° C. with oxygen or an oxygen-containing gas, thereby obtaining hot gases and a solid product,
cooling the hot gases and the solid product, and
separating off the solid product from the gases,
wherein said solution 1 comprises an oxidizable zinc compound, and said solution 2 comprises an oxidizable cerium compound,
wherein a fraction of solution 1 is 80 to 98% by weight, calculated as ZnO, and a fraction of solution 2 is 2 to 20% by weight, calculated as CeO₂,
wherein solution 1 has a viscosity of from 200 to 5000 mPas and solution 2 has a viscosity of from 5 to 150 mPas, and optionally, the viscosity is regulated by heating to temperatures in each case within the decomposition temperature of the zinc compound and of the cerium compound.

9. The process according to claim 8, wherein the reaction temperature is produced by a flame which results from the reaction of a hydrogen-containing combustion gas with oxygen and/or air.

10. A dispersion, comprising: the composite particles according to claim 1.

11. A coating composition, comprising: the composite particles according to claim 1 or the dispersion according to claim 11 and at least one binder.

12. A sunscreen formulation, comprising: the composite particles according to claim 1 or the dispersion according to claim 11.

13. An UV filter, comprising:
the composite particles according to claim 1.

14. The composite particles according to claim 2, wherein the zinc oxide fraction is 85 to 95% by weight and the cerium oxide fraction is 5 to 15% by weight, in each case based on the composite particles.

15. The composite particles according to claim 2, wherein the fraction of zinc oxide and cerium oxide is at least 99.9% by weight, based on the composite particles.

16. The composite particles according to claim 2, wherein the BET surface area is 20 to 50 m²/g.

17. The composite particles according to claim 2, wherein the matrix has an average diameter of from 20 to 100 nm.

18. The composite particles according to claim 2, wherein the domains have an average diameter of from 2 to 10 nm.

19. A dispersion, comprising: the composite particles according to claim 2.

20. A coating composition, comprising: the composite particles according to claim 2 or the dispersion according to claim 19 and at least one binder.

21. An UV filter, comprising:
the composite particles according to claim 2.
